(19) United States
(12) Patent Application Publication
(10) Pub. No.: US 2014/0216453 A1
(43) Pub. Date: Aug. 7, 2014

(54) OXYGEN CONCENTRATOR SUPPLY LINE OBERPRESSURE PROTECTION

(71) Applicant: Douglas Adam Whitcher, Atlanta, GA (US)

(72) Inventor: Douglas Adam Whitcher, Atlanta, GA (US)

(73) Assignee: KONINKLIJKE PHILIPS N.V., EINDHOVEN (NL)

(21) Appl. No.: 14/343,398

(22) PCT Filed: Sep. 5, 2012
(86) PCT No.: PCT/IB2012/054568

§ 371 Date: Mar. 7, 2014

Related U.S. Application Data
(60) Provisional application No. 61/533,912, filed on Sep. 13, 2011.

(51) Int. Cl. A61M 16/10; A61M 16/00; A61M 16/08; A61M 16/20

(52) U.S. Cl. CPC A61M 16/101; A61M 16/202; A61M 16/0003; A61M 16/209; A61M 16/0875; A61M 16/0816; A61M 2016/0027
USPC 128/202.26

(57) ABSTRACT

A portable oxygen concentrator (10) including a reservoir (26) for storing oxygen-enriched gas and a delivery line (41) for delivering the oxygen-enriched gas from the reservoir to a subject. An oxygen delivery valve (36) communicates with the reservoir via the delivery line. A sensor (48) is in communication with gas flowing through the delivery line and generates a signal related to a breathing characteristic of the subject. A controller (21) operates in a first mode wherein the controller opens the oxygen delivery valve for continuous delivery of the gas to the subject and a second mode wherein the controller selectively opens and closes the oxygen delivery valve responsive to the signal of the sensor to deliver the gas in pulsed durations. A relief valve (46) is associated with the delivery line and opens responsive to the pressure within the delivery line exceeding a predetermined threshold so as to decrease pressure within the delivery line.

This patent application claims the priority benefit under 35 U.S.C. §119(e) of U.S. Provisional Application No. 61/533,912 filed on Sep. 13, 2011, the contents of which are herein incorporated by reference.

### BACKGROUND OF THE INVENTION

1. Field of the Invention

The present disclosure pertains to pressure relief in the oxygen supply line used in oxygen concentrators.

2. Description of the Related Art

Oxygen concentrators are used to provide supplemental oxygen to improve the comfort and/or quality of life of subjects. Oxygen concentrators may be stationary and may include oxygen lines in hospitals or other facilities that provide oxygen to subjects. Oxygen concentrators may also be portable to provide ambulatory subjects with oxygen while away from the stationary systems.

Oxygen concentrators typically contain pressure transducers that are capable of detecting vacuum levels induced on the cannula line by a subject's inhalation to determine the onset of the subject's inhalation. The detection of inhalation is used to trigger the concentrator's supply line/circuit to deliver a bolus of oxygen during a pulse delivery mode wherein the oxygen is delivered to the subject at pulsed durations. For concentrators that also have a continuous delivery mode, in which oxygen is delivered to the subject continuously, the pressure sensor may be exposed to the full system pressure of the oxygen concentrator during the continuous delivery mode. The higher output concentrators that have both the continuous and pulsed delivery modes may have delivery lines that have pressure exceeding a certain threshold. However, the typical pressure transducer is not configured to allow for continuous exposure to pressures above the threshold. This limits the way in which the pneumatic circuitry of the supply line can be arranged for concentrators that implement both pulse and continuous flow delivery modes.

### SUMMARY OF THE INVENTION

Accordingly, it is an aspect of one or more embodiments of the present disclosure to provide a portable oxygen concentrator including a reservoir configured to store oxygen-enriched gas and a delivery line configured to deliver the oxygen-enriched gas from the reservoir to a subject. The concentrator also includes an oxygen delivery valve communicates with the reservoir via the delivery line and a sensor in fluid communication with gas flowing through the delivery line and configured to generate an output signal conveying information related to a breathing characteristic of the subject. The concentrator further includes a controller configured to operate in 1) a first mode wherein the controller opens the oxygen delivery valve for continuous delivery of the gas through the delivery line to the subject and 2) a second mode wherein the controller selectively opens and closes the oxygen delivery valve responsive to the output signal of the sensor to deliver the gas to the subject in pulsed durations. The concentrator also includes a relief valve associated with the delivery line and configured to open responsive to the pressure within the delivery line exceeding a predetermined threshold so as to decrease pressure within the delivery line.

It is yet another aspect of one or more embodiments of the present disclosure to provide a method for concentrating oxygen including providing a portable apparatus that includes a reservoir configured to store oxygen-enriched gas, a delivery line configured to deliver the oxygen-enriched gas from the reservoir to a subject, an oxygen delivery valve communicating with the reservoir via the delivery line; a sensor in fluid communication with gas flowing through the delivery line; a controller to control operations of the oxygen delivery valve; and a relief valve associated with the delivery line. The method also includes generating an output signal, via the sensor, conveying information related to a breathing characteristic of the subject and operating, via the controller, in (a) a first mode wherein the controller opens the oxygen delivery valve for continuous delivery of the gas to the subject or (b) a second mode wherein the controller selectively opens and closes the oxygen delivery valve responsive to the output signal of the sensor to deliver the gas to the subject in pulsed durations. The method further includes opening the relief valve responsive to the pressure within the delivery line exceeding a predetermined threshold so to decrease pressure within the delivery line.

It is yet another aspect of one or more embodiments of the present disclosure to provide a portable oxygen concentrator that includes means for storing oxygen-enriched gas and means for delivering the oxygen-enriched gas from the reservoir to a subject. The concentrator also includes an oxygen valve means for permitting or preventing oxygen enriched gas to flow through the delivery line and means for generating an output signal conveying information related to a breathing characteristic of the subject. The generation of the output signal is provided by a sensor. The concentrator also includes means for controlling operations in (a) a first mode wherein the controller opens the oxygen delivery valve for continuous delivery of the gas to the subject (b) a second mode wherein the controller selectively opens and closes the oxygen delivery valve responsive to output signal of the sensor to deliver the gas to the subject in pulsed durations. The concentrator further includes relief valve means for decreasing pressure within the delivery line responsive to the pressure within the delivery line exceeding a predetermined threshold.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1a is a perspective view of a housing member of a portable oxygen concentrator and one side of the support member supporting components of the portable oxygen concentrator;

FIG. 1b is another perspective view of a housing member and the support member of the portable oxygen concentrator in accordance with an embodiment of the present disclosure;

FIG. 2 schematically illustrates the portable oxygen concentrator in accordance with an embodiment of the present disclosure;

FIG. 3 is a cross sectional view of an embodiment of a cannula and relief valve of the portable oxygen concentrator; and

FIG. 4 is a cross sectional view of an embodiment of the relief valve of the portable oxygen concentrator.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

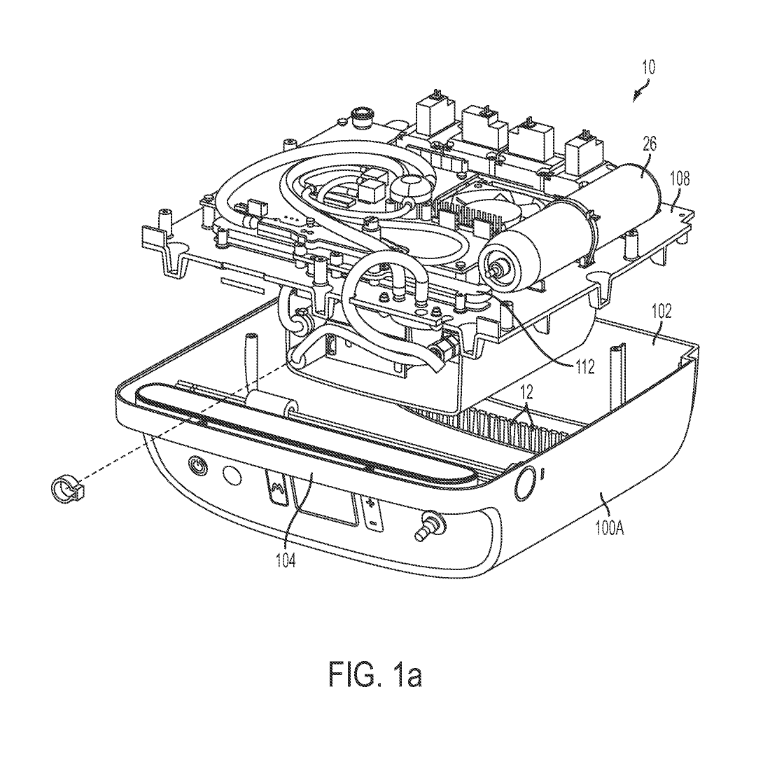

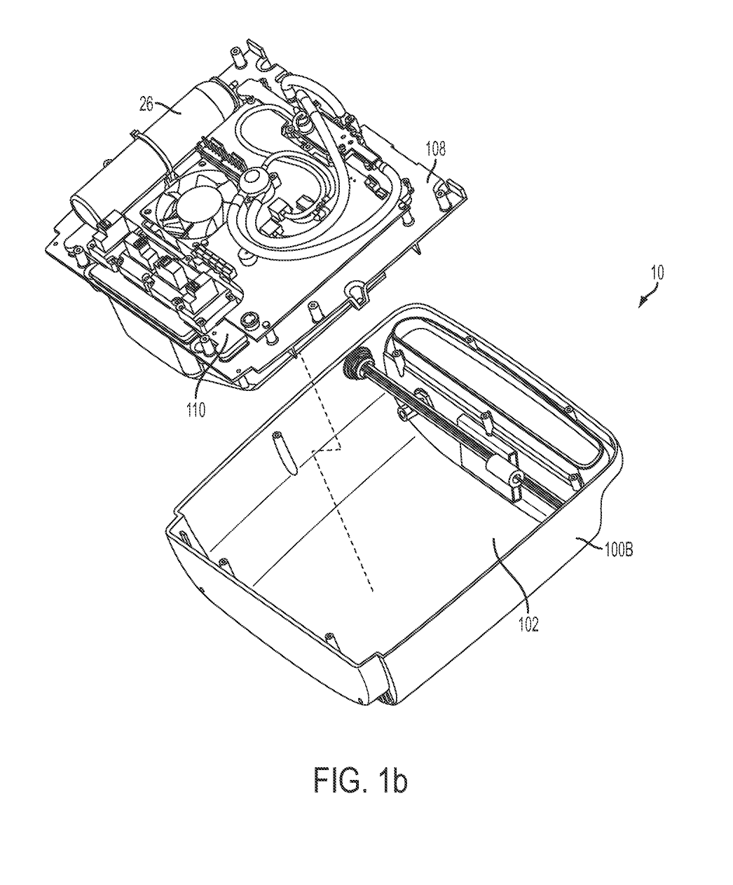

FIGS. 1a and 1b illustrate an embodiment of a portable oxygen concentrator 10 having a housing 100 formed from two mating house members 100A, 100B cooperating with each other to define a hollow interior 102 therein. Hollow interior 102 of housing 100 may house a support member 108, which supports components of portable oxygen concentrator 10. Portable oxygen concentrator 10 may include a carrying handle 104 connected to at least one of the walls to enable portable oxygen concentrator 10 to be transported.

Housing 100 may include one or more inlet openings 12 that may communicate with interior 102 of portable oxygen concentrator 10. Inlet openings 12 are configured to allow air to pass easily through inlet openings 12, yet preventing large objects from passing therethrough.

As shown in FIGS. 1a and 1b, portable oxygen concentrator 10 may include a support member (central chassis or spine) 108. An air manifold 110 and an oxygen delivery manifold 112 of portable oxygen concentrator 10 are integrally formed or integrally molded on support member 108. Manifolds 110, 112 may contain pathways or passages for air or oxygen to travel through the concentrator, which will be described in more detail later. Additional information on an exemplary central chassis or spine with integrally formed air manifold and oxygen delivery manifold may be found in U.S. provisional patent application No. 61/533,962, filed Sep. 13, 2011, the entire disclosure of which is expressly incorporated by reference herein.

Manifolds 110, 112 may be substantially rigid, e.g., thereby providing or enhancing a structural integrity of apparatus 10. The air manifold may be formed from any engineering grade material, e.g., plastic, such as ABS, polycarbonate, and the like; metal, such as aluminum, and the like; or composite materials. The air manifold may be formed by injection molding, casting, machining, and the like.

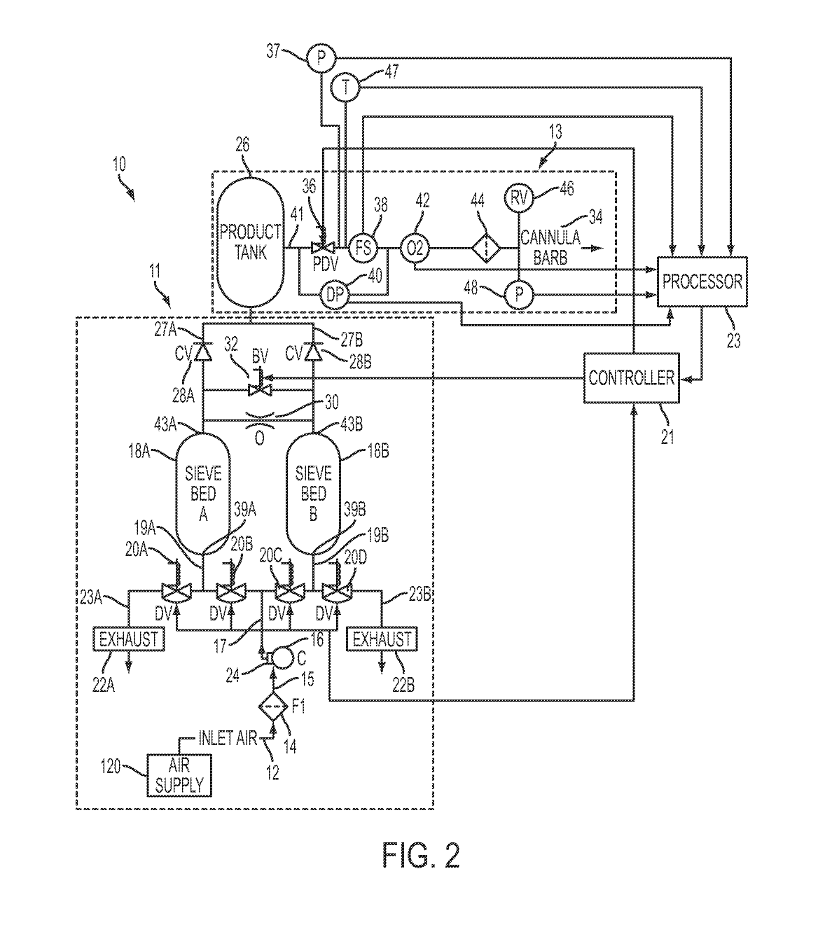

FIG. 2 is a schematic representation of an embodiment of portable oxygen concentrator 10 having an oxygen generating system 11 and an oxygen delivery system 13. Air may enter concentrator 10 through an opening 12 of concentrator 10 from an air supply 120, such as ambient air. Opening 12 may be a single opening or may be a plurality of openings. Oxygen generating system 11 includes an inlet filter 14 that is provided inline between inlet port 12 and a compressor 16 to remove dust or other particles from the ambient air drawn into inlet port 12 before it enters compressor 16. The filtered air may be communicated from filter 14 to an opening 24 of compressor 16 via a compressor passage 15. Compressor 16 is configured to compress or pressurize the air to a desired pressure level. In some embodiments, concentrator 10 may emit a high level of noise, which may primarily originate from an air inlet opening, which may be any inlet or opening that receives air from an air supply, such as ambient air, for pressurization by compressor 16.

An inlet opening restrictor (not shown), which is described in U.S. provisional patent application No. 61/533,864, filed Sep. 13, 2011, which is incorporated herein in its entirety, may be provided to dynamically change the size or shape or other characteristic of the inlet opening proportionately for all input/output settings so as to minimize the noise output for a particular setting. In one embodiment, the inlet opening may be formed on a housing of the air filter 14 and the inlet opening restrictor may be pivoted relative to the inlet opening so as to change a characteristic of the inlet opening to enable air to pass therethrough and to minimize the sound level output from the inlet opening.

Referring back to FIG. 2, oxygen generating system 11 includes diaphragm valves 20. Although four diaphragm valves (20A, 20B, 20C, and 20D) are shown in this embodiment, it should be appreciated that the number of diaphragm valves may vary in other embodiments. A controller 21 may be coupled to air control valves 20 for selectively opening and closing air control valves 20 to control airflow therethrough, and consequently, through sieve bed passages 19A, 19B to sieve beds 18A, 18B. Sieve bed passages 19A, 19B may be at least partially defined by pathways in air manifold 110.

Air control valves 20 may be selectively opened and closed to provide flow paths, e.g., from compressor 16 to sieve bed 18A, 18B through a compressor outlet passage 17 and/or from sieve bed 18A, 18B through exhaust passages 23A, 23B to exhaust ports 22A, 22B. Accordingly, when supply air control valve 20B is open, a flow path may defined from compressor 16, through compressor passage 17, through air control valve 20B, through sieve bed passage 19A, and into sieve bed 18A. When exhaust air control valve 20D is open, a flow path may be defined from sieve bed 18B, through sieve bed passage 19B, through air control valve 20D, through an exhaust passage 23B, and out exhaust opening(s) 22A, 22B.

An exemplary two-way valve that may be used for each of valves 20 is the SMC DXT valve, available from SMC Corporation of America, of Indianapolis, Ind. The valve may be provided as “normally open.” When pressure is applied to the top side of the diaphragm through the pilot valve, the diaphragm may be forced down onto a seat, shutting off the flow. Either a normally open or normally closed pilot solenoid valve may be used. Since the diaphragm valve itself is normally open, using a normally open solenoid valve may create normally closed overall operation, requiring application of electrical energy to open the valve.

In the embodiment shown in FIG. 2, oxygen generating system 11 includes at least one sieve bed 18A, 18B (two are shown in this embodiment) containing molecular sieve material configured to separate the pressurized air into a concentrated gas component for delivery to a subject. Sieve beds 18A, 18B may include a first port 39A, 39B, respectively, configured to receive air and transfer nitrogen and a second port 43A, 43B, respectively, configured to transfer oxygen out of sieve beds 18A, 18B. The sieve material may include one or more known materials capable of adsorbing nitrogen from pressurized ambient air, thereby allowing oxygen to be bled off or otherwise evacuated from sieve beds 18A, 18b. Exemplary sieve materials that may be used include synthetic zeolite, LiX, and the like, such as UOP Oxysiv 5, 5A, Oxysiv MDX, or Zeochem Z10-06. Although two sieve beds 18A, 18B are shown in FIG. 2, it will be appreciated that one or more sieve beds may be provided, e.g., depending upon the desired weight, performance efficiency, and the like.

Sieve bed 18A, 18B may be purged or exhausted, i.e., first end 39A, 39B may be exposed to ambient pressure, once the pressure within sieve bed 18A, 18B reaches a predetermined limit (or after a predetermined time). This causes the compressed nitrogen within sieve bed 18A, 18B to escape through first end 39A, 39B and to exit exhaust ports 22A, 22B. Optionally, as sieve bed 18A, 18B is being purged, oxygen escaping from other sieve bed 18A, 18B (which may be being charged simultaneously) may pass through a purge orifice 30 into second port 43A, 43B of purging sieve bed 18A, 18B, e.g., if the pressure within the charging sieve bed is greater than within the purging sieve bed, which may occur towards the end of purging. In addition or alternatively, oxygen may pass through check valves 28A, 28B located between sieve beds 18A, 18B, e.g., when the relative pressures of sieve beds 18A, 18B and reservoir 26 causes check valves 28A, 28B to open, in addition to or instead of through purge orifice 30.

Oxygen generating system 11 is configured operate sieve beds 18A, 18B such that they are alternatively “charged” and “purged” to generate concentrated oxygen. When a sieve bed 18A or 18B is being charged or pressurized, compressed ambient air is delivered from compressor 16 into first end 39A, 39B of sieve bed 18A, or 18B, causing sieve material to adsorb more nitrogen than oxygen as sieve bed 18A or 18B is pressurized. While the nitrogen is substantially adsorbed by the sieve material, oxygen escapes through second ends 43A, 43B of sieve bed 18A or 18B, where it may be stored in reservoir 26 and/or be delivered to the subject.

Exhaust ports 22A, 22B may be configured to expel exhaust air (generally concentrated nitrogen) from sieve beds 18A, 18B. In one embodiment, the exhaust air may be directed towards controller 21 or other electronics within concentrator 10, e.g., for cooling the electronics.

As further shown in FIG. 2, a purge orifice 30 may be provided between sieve beds 18A, 18B. Purge orifice 30 may remain continuously open, thereby providing a passage for oxygen to pass from one sieve bed 18A, 18B to the other, e.g., while one sieve bed 18A, 18B is charging and the other is purging. Purge orifice 30 may have a precisely determined cross-sectional size, which may be based upon one or more flow or other performance criteria of sieve beds 18A, 18B. For example, the size of purge orifice 30 may be selected to allow a predetermined oxygen flow rate between the charging and purging sieve beds 18A, 18B. It is generally desirable that the flow through purge orifice 30 is equal in both directions, such that both sieve beds 18A, 18B may be equally purged, e.g., by providing a purge orifice 30 having a geometry that is substantially symmetrical.

Oxygen generating system 11 may also include an oxygen side balance valve 32 between sieve beds 18A, 18B configured to balance bed pressures in sieve bed 18A and sieve bed 18B so as to maximize efficiency (e.g., to reduce power consumption). During the pressure cycling of sieve beds 18A, 18B, the pressure in sieve bed 18A may be higher than the pressure in sieve bed 18B indicating that the beds are not balanced. In such an instance, balance valve 32 is operated (opened) to relieve some pressure from sieve bed 18A and provide the pressure to sieve bed 18B, for example, before compressor 16 switches from sieve bed 18A to sieve bed 18B to supply compressed air to sieve bed 18B. Transferring some pressure from sieve bed 18A to sieve bed 18B allows sieve bed 18B be at some intermediate pressure (rather than be at a zero pressure), when compressor starts supplying compressed air to sieve bed 18B.

As mentioned above, check valves 28A, 28B may open to enable oxygen to pass therethrough. Check valves 28A, 28B may simply be pressure-activated valves that provide one-way flow paths from sieve beds 18A, 18B of oxygen generating system 11 into reservoir 26 of oxygen delivery system 13 through oxygen delivery passages 27A, 27B. Oxygen delivery passage 27A, 27B may be at least partially defined by pathways in oxygen manifold 112. Because check valves 28A, 28B allow one-way flow of oxygen from sieve beds 18A, 18B into reservoir 26 and oxygen delivery passages 27A, 27B, whenever the pressure in either sieve bed 18A, 18B exceeds the pressure in reservoir 26, the respective check valve 27A, 27B may open. Once the pressure within either sieve bed 18A, 18B becomes equal to or less than the pressure in reservoir 26, the respective check valve 28A, 28B may close.

Oxygen delivery system 13 includes reservoir 26 that stores oxygen enriched gas and a connection portion 34 (e.g., a cannula barb) that connects to a subject interface (e.g., a cannula) for delivery of the oxygen to the subject. In an alternative embodiment, concentrator 10 may include multiple reservoirs (not shown) that may be provided at one or more locations within concentrator 10. Concentrator 10 may also include one or more flexible reservoirs, e.g., bags or other containers that may expand or contract as oxygen is delivered into or out of them. The reservoirs may have predetermined shapes as they expand or more expand elastically to fill available space within concentrator 10. Optionally, one or more rigid reservoirs may be provided that communicate with one or more flexible reservoirs (not shown), e.g., to conserve space within concentrator 10.

In one embodiment, oxygen delivery system 13 includes a delivery or supply line 41 with a proportional oxygen delivery valve 36, a flow sensor 38, a local pressure sensor 37, an oxygen gas temperature sensor 47, a pressure sensor 40, an oxygen sensor 42, a filter 44, a relief valve 46, and a pressure sensor 48 associated therewith. Delivery line 41 may also include an external cannula line (not shown) configured to connect to a cannula to deliver oxygen to the subject. These components may be of the same type as described in U.S. provisional patent application No. 61/533,871, filed Sep. 13, 2011, which is incorporated herein in its entirety. In this embodiment, delivery line 41 is used to deliver oxygen to the subject during continuous mode and pulse delivery mode.

Oxygen delivery valve 36 may be configured to control the flow of oxygen through an oxygen delivery passage or line 41 from reservoir 26 out of concentrator 10 to a subject. Oxygen delivery valve 36 may be a solenoid valve coupled to controller 21 that may be selectively opened and closed. An exemplary valve that may be used for oxygen delivery valve 36 is the Hargraves Technology Model 45M, which may have a relatively large orifice size, thereby maximizing the possible flow through oxygen delivery valve 36. Alternatively, it may also be possible to use a Parker Pneutronics V Squared or Series 11 valve. Controller 21 may be configured to control when proportional oxygen delivery valve 36 is fully open, fully closed, or partially open as well as the degree to which valve 36 is open based on the received inputs from the sensors. When oxygen delivery valve 36 is open, oxygen may flow through oxygen delivery passage 41 and through oxygen delivery valve 36 to the subject. Oxygen delivery valve 36 may be opened for desired durations at desired frequencies, which may be varied by controller 21, thereby providing pulse delivery. Alternatively, controller 21 may maintain oxygen delivery valve 36 open to provide continuous delivery, rather than pulsed delivery. In this alternative, controller 21 may throttle oxygen delivery valve 36 to adjust the volumetric flow rate to the subject.

Pressure sensor 40 may be coupled to processor 23, e.g., to provide signals that may be processed by processor 23 to determine the pressure differential across oxygen delivery valve 36. Controller 21 may use this pressure differential to determine a flow rate of the oxygen being delivered from portable oxygen concentrator 10 or other parameters of oxygen being delivered. Controller 21 may change the frequency and/or duration that oxygen delivery valve 36 is open based upon the resulting flow rates, e.g., based upon one or more feedback parameters.

Flow sensor 38 may also be coupled to processor 23 and configured to measure the instantaneous mass flow of the oxygen passing through delivery line 41 and to provide feed-back to proportional oxygen delivery valve 36. In one embodiment, flow sensor 38 is a mass flow sensor. Use of piezo-electric proportional valve 36 with closed loop (feed-back) control via mass flow sensor 38 allows portable oxygen concentrator 10 to deliver oxygen in either continuous flow or pulse flow waveforms. This arrangement also allows portable oxygen concentrator 10 to use a single delivery valve 36 or circuit to deliver both continuous flow and pulse flow waveforms of dynamically controllable flow and delivery time.

Oxygen gas temperature sensor 47 is configured to measure the temperature of the oxygen passing through delivery line 41, while local pressure sensor 37 is configured to measure the local ambient pressure.

The measured oxygen temperature and the measured local ambient pressure are sent to a processor 23. Processor 23 is configured to use this oxygen temperature measurement from temperature sensor 47 and the local ambient pressure measurement from local pressure sensor 37 along with the mass flow rate measurement obtained from flow sensor 38 to obtain a volumetric flow rate measurement. Oxygen gas temperature sensor 47 and local pressure sensor 37 may be positioned upstream of flow sensor 38. In another embodiment, oxygen gas temperature sensor 47 and local pressure sensor 37 may be positioned downstream (still in the vicinity) of flow sensor 38.

Oxygen sensor 42 may be coupled to processor 23 and may generate electrical signals proportional to the purity that may be processed by controller 21 and used to change operation of the concentrator 10. Because the accuracy of oxygen sensor 42 may be affected by airflow therethrough, it may be desirable to sample the purity signals during no flow conditions, e.g., when proportional oxygen delivery valve 36 is closed.

Processor 23 of portable oxygen concentrator 10 may be configured to receive the signals from one or more sensing components of portable oxygen concentrator 10, e.g., flow sensor 38, pressure sensor 40, oxygen sensor 42 and/or pressure sensor 48, to determine a flow of the oxygen-enriched gas in the delivery line over a predetermined period of time, a volume of the oxygen-enriched gas in the delivery line over a predetermined period of time or both based on the received signal.

Air filter 44 may include any conventional filter media for removing undesired particles from oxygen being delivered to the subject. Air filter 44 may be provided either downstream or upstream of relief valve 46 and pressure sensor 48.

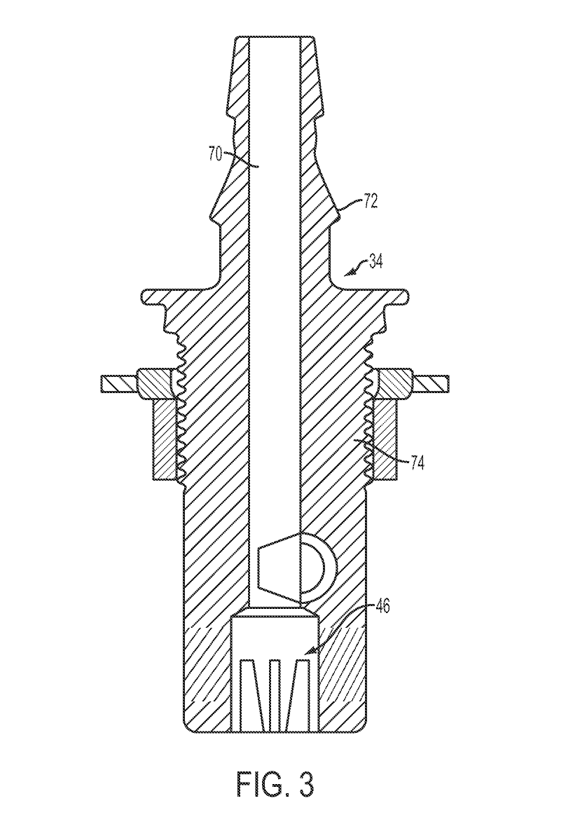

Relief valve 46 is configured to relieve pressure (open) responsive to the pressure within delivery line 41 exceeding a predetermined threshold so as to decrease pressure within delivery line 41 when oxygen is supplied to the subject. Although relief valve 46, as shown in FIG. 2, is located between cannula barb 34 and air filter 44, it should be appreciated that relief valve 46 may be located elsewhere on delivery line 41 so long as relief valve 36 is in communication with the gas flowing through delivery line 41. For example, relief valve 36 may be connected to an internal tubing that leads to cannula barb 34, may be attached to cannula barb 34 (as shown in FIG. 3), or may be connected to an external cannula line.

In the embodiment shown in FIG. 3, relief valve 46 may be located within cannula barb 34. Cannula barb 34 may include a connection portion 72 configured to be connected to an external cannula line or any other conduit that communicates oxygen to the subject. Cannula barb 74 may also include a concentrator portion 74 configured to be connected to concentrator 10. A passage 70 may be provided in cannula barb 70 to enable oxygen to flow therethrough. Relief valve 46 may be in communication with the oxygen flowing through passage 70.

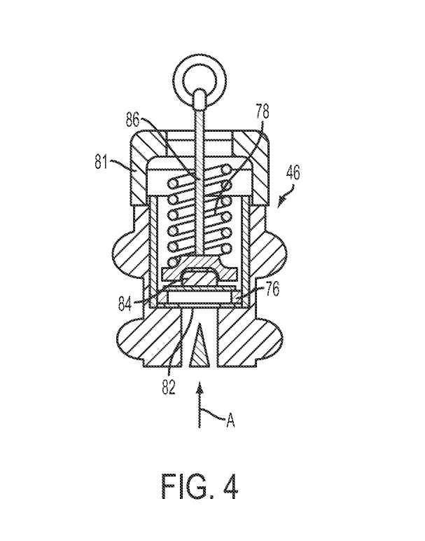

FIG. 4 shows an embodiment of relief valve 46 taking the form of a normally closed mechanical poppet valve. Relief valve 46 may be in a closed position wherein oxygen is prevented from being passed therethrough and an open position wherein oxygen is permitted to pass therethrough. Relief valve 46 may include a housing 81 having an opening 82 for oxygen to flow into valve 36, a spring 78 disposed inside housing 81, a poppet 84 for closing and opening valve 36, a stem 86 that is connected to poppet 84, and a seat 86 that contacts poppet 84 when the valve is in the closed position. Relief valve 46 may also include outlets (not shown) for oxygen to pass therethrough to decrease the pressure inside the delivery line 41 when the valve 46 is in the open position. Spring 78 may normally push the poppet 84 in a closed position against the seat 76 so as to seal opening 82 of housing 81 to prevent oxygen from flowing into valve 46.

The pressure of the oxygen may push poppet 84 away from seat 76, thus moving valve 46 to the open position. That is, spring 78 is configured to oppose movement of poppet 84 in the direction of A, while the pressure of the oxygen may push poppet 84 in the direction of A. Seat 76 may be made of elastomeric material that enables poppet 84 to form a seal with seat 76 so as to prevent oxygen from flowing therein. The characteristics of the spring, such as the spring force and/or elasticity, may be varied according to the desired predetermined threshold pressure at which valve 46 may be opened. That is, the desired predetermined threshold at which valve 46 may open when the pressure in the delivery line 41 exceeds the threshold may be associated with the force of the spring. The force of the spring may be varied based on, for example, Hooke's law.

It should be appreciated that relief valve 46 may have other configurations or take other forms in other embodiments. Relief valve 46 may be a mechanical valve, but may also be an electronic valve operated by the controller 21 in some embodiments. For example, relief valve 46 may be a normally closed pilot solenoid valve configured to be opened by controller 21 when controller 21 determines that the pressure within delivery line 41 is above or at a certain threshold. However, it should be appreciated that these examples are not intended to be limiting and relief valve 46 may have other configurations in other embodiments.

Referring back to FIG. 2, pressure sensor 48 may be in fluid communication with gas flowing through delivery line 41 and may be used during pulse delivery mode. Sensor 48 may be configured to generate an output signal conveying information related to a breathing characteristic of the subject. For example, pressure sensor 48 may be configured to sense the pressure within delivery line 41 so that inhalation of the subject may be detected. The subject breathing rate may be determined by controller 21, e.g., based upon pressure readings from pressure sensor 48. Pressure sensor 48 may detect a reduction in pressure as the subject inhales.

Controller 21 may monitor the frequency at which pressure sensor 48 detects the reduction in pressure to determine the breathing rate. In addition, controller 21 may also use the pressure differential detected by pressure sensor 48. Pressure sensor 122 may measure an absolute pressure of the oxygen within delivery line 41. This pressure reading may be used to detect when a subject is beginning to inhale, e.g., based upon a resulting pressure drop within delivery line 41, which may trigger delivering a pulse of oxygen to the subject, which will be described in more detail later. Because pressure sensor 48 may be exposed to the full system pressure of concentrator 10, it may be desirable for the over-pressure rating of pressure sensor 48 to exceed the full system pressure.

Pressure sensor 48 may be a piezo resistive pressure sensor capable of measuring absolute pressure. Exemplary transducers that may be used include the Honeywell Microswitch 24PC01SMT Transducer, the Sensym SX01, Motorola MOX, or others made by All Sensors. Because pressure sensor 48 may be exposed to the full system pressure of concentrator 10, it may be desirable for the over-pressure rating of pressure sensor 48 to exceed the full system pressure.

Controller 21 may include one or more hardware components and/or software modules that control one or more aspects of the operation of portable oxygen concentrator 10. Controller 21 may be coupled to one or more components of portable oxygen concentrator 10, e.g., compressor 16, air control valves 20, and/or oxygen delivery valve 36. Controller 21 may also be coupled to one or more components of oxygen concentrator 10, such as the sensors, valves, or other components. The components may be coupled by one or more wires or other electrical leads capable of receiving and/or transmitting signals between controller 21 and the components.

Controller 21 may also be coupled to a subject interface (not shown), which may include one or more displays and/or input devices. The subject interface may be a touch-screen display that may be mounted to portable oxygen concentrator 10. The subject interface may display information regarding parameters related to the operation of portable oxygen concentrator 10 and/or allow the subject to change the parameters, e.g., turn portable oxygen concentrator 10 on and off, change dose setting or desired flow rate, etc. Portable oxygen concentrator 10 may include multiple displays and/or input devices, e.g., on/off switches, dials, buttons, and the like (not shown). The subject interface may be coupled to controller 21 by one or more wires and/or other electrical leads (not shown for simplicity), similar to the other components.

Controller 21 may include a single electrical circuit board that includes a plurality of electrical components thereon. These components may include one or more processors 23, memory, switches, fans, battery chargers, and the like (not shown) mounted to the circuit board. It will be appreciated that controller 21 may be provided as multiple subcontrollers that control different aspects of the operation of portable oxygen concentrator 10. For example, a first subcontroller may control operation of compressor 16 and the sequence of opening and closing of air control valves 20, e.g., to charge and purge sieve beds 12 in a desired manner. Additional information on an exemplary first subcontroller that may be included in portable oxygen concentrator 10 may be found in U.S. Pat. No. 7,794,522, the entire disclosure of which is expressly incorporated by reference herein.

In the embodiment shown in FIG. 2, oxygen delivery system 13 includes delivery line 41 that is capable of selectively delivering oxygen in a pulsed or continuous manner Thus, the concentrator 10 includes a first mode, or continuous mode wherein controller 21 opens oxygen delivery valve 36 for continuous delivery of the oxygen through delivery line 41 to the subject and a second mode, or pulsed delivery mode, wherein controller 21 selectively opens and closes valve 36 responsive to the output signal of pressure sensor 48 to deliver the oxygen through delivery line 41 to the subject in pulsed durations.

Controller 21 may open oxygen delivery valve 36 after controller 21 detects an event, such as detecting when the subject begins to inhale via pressure sensor 48. When the event is detected, oxygen delivery valve 36 may be opened for the predetermined pulse duration. In this embodiment, the pulse frequency or spacing (time between successive opening of oxygen delivery valve 36) may be governed by and correspond to the breathing rate of the subject (or other event spacing). The overall flow rate of oxygen being delivered to the subject is then based upon the pulse duration and pulse frequency.

Optionally, controller 21 may delay opening oxygen delivery valve 36 for a predetermined time or delay after detection of subject inhalation via pressure sensor 48, e.g., to maximize delivery of oxygen to the subject. For example, this delay may be used to maximize delivery of oxygen during the “functional” part of inhalation. The functional part of the inhalation is the portion where most of the oxygen inhaled is absorbed into the bloodstream by the lungs, rather than simply used to fill anatomical dead space, e.g., within the lungs. It has been found that the functional part of inhalation may be approximately the first half and/or the first six hundred milliseconds (600 ms) of each breath. Thus, it may particularly useful to detect the onset of inhalation early and begin delivering oxygen quickly in order to deliver oxygen during the functional part of inhalation.

In one embodiment, controller 21 may include hardware and/or software that may filter the signals from pressure sensor 48 to determine when the subject begins inhalation. In this alternative, controller 21 may need to be sufficiently sensitive to trigger oxygen delivery valve 36 properly, e.g., while the subject employs different breathing techniques. In one embodiment, controller 21 may open at a pulse frequency that may be fixed, i.e., independent of the subject's breathing rate, or that may be dynamically adjusted. For example, controller 21 may open oxygen delivery valve 36 in anticipation of inhalation, e.g., based upon monitoring the average or instantaneous spacing or frequency of two or more previous breaths. In a further alternative, controller 21 may open and close oxygen delivery valve 36 based upon a combination of these parameters.

The subject breathing rate may be determined by controller 21, e.g., based upon pressure readings from pressure sensor 48. Pressure sensor 48 may detect a reduction in pressure as the subject inhales. Controller 21 may monitor the frequency at which pressure sensor 48 detects the reduction in pressure to determine the breathing rate. In addition, controller 21 may also use the pressure differential detected by pressure sensor 40.

For pulse delivery, the pulse duration may be based upon a dose setting selected by the subject. In this way, substantially the same volume of oxygen may be delivered to the subject each time oxygen delivery valve 36 is opened, given a specific dose setting. The dose setting may be subject selected or predetermined. In one embodiment, the dose setting may include a quantitative and/or qualitative setting. Controller 21 may relate the subject-selected qualitative setting with a desired flow rate or bolus size, e.g., relating to the maximum flow capacity of apparatus 10. The settings may correspond to points within the range at which apparatus 10 may supply concentrated oxygen. For example, a maximum flow rate (or equivalent flow rate of pure oxygen) for apparatus 10 may be used.

Alternatively, a maximum bolus volume may be used. A quantitative setting may allow a subject to select a desired flow rate, which may be an actual concentrated oxygen flow rate or an equivalent pure oxygen flow rate, or a desired bolus volume. The flow rates or volumes available for selection may also be limited by the capacity of apparatus 10, similar to the qualitative settings. As the dose setting is increased, the pulse duration may be increased to deliver a predetermined bolus during each pulse. If the subject's breathing rate remains substantially constant, the pulse frequency may also remain substantially constant, thereby increasing the overall flow rate being delivered to the subject. The flow rate may also be based upon the setting selected by the subject during continuous delivery.

As noted above, pressure sensor 48 may be configured to detect drops or reduction in pressure of the oxygen within cannula line 43 or vacuum levels induced on cannula line 43 by the subject's inhalation to determine onset of inhalation for the pulse delivery mode. In one embodiment, pressure sensor 48 may be calibrated to detect vacuum levels on the order of, for example, 0.1 inches of H₂0. Pressure sensor 48 may be exposed to the full system pressure of apparatus 10. In embodiment where portable oxygen concentrators 10 produce lower outputs, e.g., those having less than 1 liter per minute (LPM) maximum output, the pressure in reservoir 26 of concentrator 10 may be less than a certain threshold (e.g., 12 psig). Thus, pressure sensor 48 may be exposed to pressure less than the threshold (e.g., 12 psig) with the actual level of the pressure being dependent on the amount of downstream restriction from delivery valve 36 and the resulting backpressure on delivery line 41 when delivery valve 36 is open.

In embodiments of oxygen concentrator 10 that have a higher output, e.g., those that have more than 1 LPM maximum output, the pressure in reservoir 26 of concentrator 10 may exceed the threshold (e.g., 12 psig) with the actual level being dependent on the timing parameters of the PSA (pressure swing adsorption) process that is implemented. That is, the pressure within delivery line 41 may exceed the operational limits of pressure sensor 48 when concentrator 10 is operating in continuous mode. Accordingly, relief valve 46 may be configured to open responsive to the pressure within delivery line 41 exceeding a predetermined threshold so as to decrease the pressure within delivery line 41 when the concentrator is in the continuous mode. The predetermined threshold may be at or below the operational proof pressure of pressure sensor 48. Just for example, in one embodiment, pressure sensor 48 may have a proof pressure of 10 psig. In such an embodiment, the predetermined threshold may be set to 6 psig so as to provide a margin for tolerance. That is, in such an embodiment, relief valve 46 may be configured to open when the pressure is at or exceeds 6 psig. Accordingly, relief valve 46 may decrease the pressure within delivery line 41 such that the pressure is below operational proof pressure of pressure sensor 48 to protect the electronics of pressure sensor 48.

During pulsed delivery of oxygen to the subject via delivery liner 41, controller 21 may open and close delivery valve 36 according to the dose setting of concentrator 10 and the output signals generated by pressure sensor 48 indicating the breathing characteristics of the subject. Relief valve 48 may be normally closed during the pulsed delivery mode. During continuous delivery of oxygen to the subject via delivery line 41, controller 21 may maintain the delivery valve 36 in the open position to deliver the oxygen to the subject at a predetermined flow rate. Pressure sensor 48 may be continuously exposed to the pressure, which may be at or near the pressure level within reservoir 26. In addition, in some situations, such as when the external cannula line is kinked or somehow overly restricted, the pressure may increase within delivery line 41. Accordingly, the relief valve 46 may open so as to decrease the pressure within delivery line 41 to protect pressure sensor 48.

In the embodiment of the relief valve shown in FIG. 3, the pressure may push poppet 84 upwards in the direction of A against the force of spring 78 and away from seat 76, thus enabling oxygen to flow through opening 82 and into relief valve 46, which may then output the oxygen into the ambient air. After the pressure has been sufficiently decreased, spring 78 may bias poppet 84 back against seat 76 so as to close valve 46. Thus, relief valve 46 may be used to protect pressure sensor 48 when the pressure within delivery line 41 exceeds a predetermined threshold.

Portable oxygen concentrator 10 may include one or more power sources, coupled to controller 21, processor 23, compressor 16, air control valves 20, and/or oxygen delivery valve 36. For example, a pair of batteries (not shown) may be provided that may be mounted or otherwise secured to portable oxygen concentrator 10. Mounts, straps or supports (not shown) may be used to secure the batteries to portable oxygen concentrator 10. Additional information on exemplary batteries that may be included in portable oxygen concentrator 10 may be found in U.S. Pat. No. 7,794,522, the entire disclosure of which is expressly incorporated by reference herein. Controller 21 may control distribution of power from batteries to other components within portable oxygen concentrator 10. For example, controller 21 may draw power from one of the batteries until its power is reduced to a predetermined level, whereupon controller 21 may automatically switch to the other of the batteries.

Optionally, portable oxygen concentrator 10 may include an adapter such that an external power source, e.g., a conventional AC power source, such as a wall outlet, or a portable AC or DC power source, such as an automotive lighter outlet, a solar panel device, and the like (not shown). Any transformers or other components (also not shown) necessary to convert such external electrical energy such that it may be used by portable oxygen concentrator 10 may be provided within portable oxygen concentrator 10, in the cables connecting portable oxygen concentrator 10 to the external power source, or in the external device itself.

It should be appreciated that any of the passages described herein may be any type and combination of conduits, tubes, or other structures that enable air or other fluids to pass therethrough. In some embodiments, the passages may be built into the support member 108, air manifold 110, or delivery manifold 112 described in U.S. provisional patent application No. 61/533,874, filed Sep. 13, 2011, which is incorporated herein in its entirety.

It should be appreciated that the embodiment of the portable oxygen concentrator 10 described is not intended to be limiting. The portable oxygen concentrator 10 may include one or more additional components, e.g., one or more check valves, filters, sensors, electrical power sources (not shown), and/or other components, at least some of which may be coupled to controller 21 (and/or one or more additional controllers, also not shown), as described further below. It should be appreciated that the terms “airflow,” “air,” or “gas” are used generically herein, even though the particular fluid involved may be ambient air, pressurized nitrogen, concentrated oxygen, and the like.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A portable oxygen concentrator, comprising:
a reservoir configured to store oxygen-enriched gas;
a delivery line configured to deliver the oxygen-enriched gas from the reservoir to a subject;
an oxygen delivery valve communicating with the reservoir via the delivery line;
a sensor in fluid communication with the oxygen-enriched gas flowing through the delivery line and configured to generate an output signal conveying information related to a breathing characteristic of the subject;
a controller configured to operate in:
1) a first mode wherein the controller opens the oxygen delivery valve for continuous delivery of the oxygen-enriched gas through the delivery line to the subject, and
2) a second mode wherein the controller selectively opens and closes the oxygen delivery valve responsive to the output signal of the sensor to deliver the oxygen-enriched gas to the subject in pulses; and
a relief valve associated with the delivery line and configured to open and expel the oxygen-enriched gas out of the delivery line, responsive to pressure within the delivery line exceeding a predetermined threshold so as to decrease the pressure within the delivery line.

2. The portable oxygen concentrator of claim 1, wherein the sensor is a pressure transducer.

3. The portable oxygen concentrator of claim 1, further comprising a cannula barb associated with the delivery line, and wherein the relief valve is attached to the cannula barb.

4. The portable oxygen concentrator of claim 1, wherein the relief valve comprises a poppet and spring configured to open mechanically responsive to the pressure within the delivery line exceeding the predetermined threshold.

5. The portable oxygen concentrator of claim 1, further comprising a cannula barb associated with the delivery line, wherein the relief valve is connected to the delivery line between the cannula barb and the sensor.

6. A method for concentrating oxygen comprising:
providing a portable apparatus comprising: a reservoir configured to store oxygen-enriched gas, a delivery line configured to deliver the oxygen-enriched gas from the reservoir to a subject, an oxygen delivery valve communicating with the reservoir via the delivery line; a sensor in fluid communication with the oxygen-enriched gas flowing through the delivery line; a controller to control operations of the oxygen delivery valve; a relief valve associated with the delivery line;
generating an output signal, via the sensor, conveying information related to a breathing characteristic of the subject;
operating, via the controller, in (a) a first mode wherein the controller opens the oxygen delivery valve for continuous delivery of the oxygen-enriched gas to the subject or (b) a second mode wherein the controller selectively opens and closes the oxygen delivery valve responsive to the output signal of the sensor to deliver the oxygen-enriched gas to the subject in pulses;
opening the relief valve and expelling the oxygen-enriched gas out of the delivery line, responsive to pressure within the delivery line exceeding a predetermined threshold so to decrease the pressure within the delivery line.

7. The method of claim 6, wherein the sensor is a pressure transducer.

8. The method of claim 6, wherein the apparatus further comprises a cannula barb associated with the delivery line, and wherein the relief valve is attached to the cannula barb.

9. The method of claim 6, wherein the relief valve comprises a poppet and spring configured to open mechanically responsive to the pressure within the delivery line exceeding the predetermined threshold.

10. The method of claim 6, wherein the apparatus further comprises a cannula barb associated with the delivery line, wherein the relief valve is connected to the delivery line between the cannula barb and the sensor.

11. A portable oxygen concentrator, comprising:
means for storing oxygen-enriched gas;
means for delivering the oxygen-enriched gas from the reservoir to a subject;
oxygen valve means for permitting or preventing oxygen enriched gas to flow through the delivery line;
means for generating an output signal conveying information related to a breathing characteristic of the subject, the generation of the output signal being provided by a sensor;
means for controlling operations in (a) a first mode wherein the controller opens the oxygen delivery valve for continuous delivery of the oxygen-enriched gas to the subject (b) a second mode wherein the controller selectively opens and closes the oxygen delivery valve responsive to output signal of the sensor to deliver the oxygen-enriched gas to the subject in pulses; and
relief valve means for decreasing pressure within the delivery line by expelling oxygen-enriched gas out of the delivery line, responsive to the pressure within the delivery line exceeding a predetermined threshold.

12. The portable oxygen concentrator of claim 11, wherein the means for sensing is a pressure transducer.

13. The portable oxygen concentrator of claim 11, further comprising a means for connecting to a cannula, and wherein the relief valve means is attached to the means for connecting to a cannula.

14. The portable oxygen concentrator of claim 11, wherein the relief valve means comprises a poppet and spring configured to open mechanically responsive to the pressure within the delivery line exceeding the predetermined threshold.

15. The portable oxygen concentrator of claim 11, further comprising a means for connecting to a cannula, wherein the relief valve means is connected to the delivery line between the means for connecting to the cannula and the sensor.
